(19) United States
(12) Patent Application Publication
(10) Pub. No.: US 2024/0228922 A1
(43) Pub. Date: Jul. 11, 2024

(54) ALGAE CULTIVATION SYSTEM

(71) Applicant: Levar Jackson, Brooklyn, NY (US)

(72) Inventor: Levar Jackson, Brooklyn, NY (US)

(21) Appl. No.: 18/410,507

(22) Filed: Jan. 11, 2024

Related U.S. Application Data
(60) Provisional application No. 63/479,488, filed on Jan. 11, 2023.

(51) Int. Cl. C12M 1/00; C12M 1/12; C12M 1/26; C12M 1/34; C12M 1/36

(52) U.S. Cl. CPC C12M 21/02; C12M 31/10; C12M 33/04; C12M 37/00; C12M 41/12; C12M 41/26; C12M 41/30; C12M 41/48

(57) ABSTRACT

A system for algae cultivation, encompassing a set of track segments, load sites, growth lights, sensors, a processor, a heater, and a harvest site. The load sites are designed to transport algae onto the track segments, which facilitate and regulate the flow of liquid, algae, growth medium, and nutrients. The processor is configured to respond to sensor data or set thresholds by adjusting track segments, dispensing nutrients, managing pH balance, and controlling the heater and growth lights. The harvest site is equipped for separating algae from the growth medium through methods like dehydration or filtering, streamlining the harvesting process. This system offers a comprehensive approach to algae cultivation and harvesting.

### PRIORITY CLAIM

This non-provisional application claims priority to and the benefit of provisional application No. 63/479,488, filed Jan. 11, 2023, which is incorporated herein as if restated in full.

### SUMMARY

The system comprises the inclusion of an algae growth medium. The algae growth medium may comprise algae content, nutrients, and water. Exemplary mediums include BG-11 for cyabobacteria and freshwater algae and F/2 for marine algae. Nutrient solutions may be infused with nitrogen, phosphorus, and/or other elements. The nutrients may include various minerals or materials which are necessary or beneficial for algae growth. Additional supplements, such as vitamins and growth enhancers may be added. The concentrations of these elements will be selected based on the desired algae composition and may be modified by the addition of additional water or by dehydration. The pH levels, generally varying between 6.5 to 8.5, may similarly be selected and controlled based on the desired algae composition. Sterilization may be achieved through autoclaving or filtration. The algae growth medium parameters may be tested prior to the introduction of algae content.

The algae content may include one or more species or sub-species of algae, including diatom, marimo, wakame, green algae, golden algae, Irish Moss, giant kelp, hijiki, latok, sea lettuce, arame, kombu, etc. The algae content selected may be supplied via laboratory or commercial suppliers, or in-house to the technology described herein. The algae content may be entered into the medium via inoculation or similar techniques. The algae content may be pipetted and then slowly mixed into the medium, although gross industrial inoculation techniques may also be utilized.

The algae may be fermented to yield alcohol (ethanol) for drink or fuel. Algae for algae-as-alcohol may be selected for high carbohydrate and starch content, and enzymatic hydrolysis may be used to breakdown the same into simple sugars. They may be inoculated with yeast (Saccharomyces cerevisiae) to ferment the sugar into ethanol.

The algae may also be processed as a textile or as textile pulp. The algae may be processed as pigmentation or media for beauty products, including makeup, (nutrients in) moisturizer, shampoo, conditioner, etc.

The system comprises a closed loop algae-water circulation track for circulating an aquatic algae growth medium. Circulation of material through the circulation track may be achieved through gravity, sloped tracks, siphoning, static mixers, baffles, cascades, automated valves, submersible pumps, inline pumps, variable speed pumps, air lifts, diffusers, propellers or impellers, paddle wheels, and using techniques such as flow control, programmed automation of pumps, valves, and mixers, etc., pulsed flow techniques such as intermittent pumping, and hydrodynamic flow modelling software. The track may consist of pipes, tubes, or other fluid flow conveyors. The track may be open or close-top. If the track is close-top, the top may be opaque or transparent, and may be made of glass, plastic, stainless steel, or any other material identified as comprising the segments, below. A closed-top may be helpful in ensuring controlled production, but an open-top may be employed to capture atmospheric benefits.

The system may comprise an external controller. The controller may be one or more computers, and may comprise a display device and one or more input devices. The controller may be a dedicated device, or conventional desktop or laptop computer, or a mobile device such as a smart phone. The controller is configured to receive instructions from the user pertaining to various system components. The external controller may receive, display, initiate, and control sensor data detection, load site, harvesting, cycle, kill, flush, and sanitization events, schedules, programs, and parameterizations. In particular, the external controller may enable a user to view and execute environmental monitoring, automation, nutrient dosing, aeration control, flow regulation, harvesting times, connectivity and remote access (such as via Wi-Fi), cleaning and maintenance, and emergency protocols.

The system may comprise a processor. The processor may be one or more computers, and may be a dedicated device, or conventional desktop or laptop computer, or a mobile device such as a smart phone. The processor may be separate from or combined with the external controller. The processor may receive feedback from the various system sensors, and convey instructions received from the external controller and relay the instructions to various system components. The processor may be programmed to automatically transmit instructions based on sensor feedback parameters. The processor may run one or more software applications in order to process system data.

The system may comprise a load site, which is a mechanism disposed on the track and configured to load the track with the load items, such as algae growth media, algae content, nutrients, minerals, vitamins, carbon dioxide, oxygen, and water. In one variation, a plurality of load sites are disposed throughout the track. In another variation, each load site is dedicated to a sub-set of load items.

The load site mechanism may consist of one or more tangent tracks and load capsule. The tangent tracks may each be in fluid communication with the primary track, with fluid communication controlled via one or more gates. The load capsule may be a container pre-loaded with load items and configured to fit snugly into a tangent track receiving vessel. In one variation, each load site is dedicated to receiving a separate load capsule type.

The load site mechanism may be configured to release a discrete unit of resources via the load capsule, or a steady stream of resources via the fluid flow of (aerated) gas or liquid. The load site mechanisms for fluid flow may consist of nozzle matrices distributed through the track or track segments. The resources for either the load capsules or non-discrete loading may be stored in dedicated or mixed reservoirs, with these reservoirs having their own sensors and load sites to prep the resources for content and concentration.

The load site mechanism may be in communication with the processor, such that loading occurs upon receiving instructions from the processor.

The system may comprise sets of live or dormant algae contained in load capsules or in reservoirs, and then upon receiving instructions pertaining to a given species of algae, load the designated algae at the load site. The load capsules may be designated using a code and may be stored in a matrix so that similar conditions may be applied to algae in load capsules throughout the load capsule matrix. In one embodiment, the load capsule matrix may be or disposed on a rotating disc or gear, with the load capsules occupying positions on the circumference or spokes on the gear, thereby enabling a designated load capsule to engage with the load site rotation of the disc or gear. In another embodiment, the load capsule matrix is disposed on a rail, with the rail configured to slide or otherwise move along an axis adjacent to the load site in order to allow for closer adjacency of the designated load capsule to the load site. In yet another embodiment, the load matrix is a grid capable of moving vertically and horizontally to provide for engagement between the load site and the designated load capsule. In a grid like matrix, the load capsules may be organized categorically, with a given row dedicated to load capsules carrying algae content, another given row dedicated to load capsules carrying algae medium, etc.

The load capsules may be containers with an opening on one side. The opening may feature a frame configured to lock onto an opposing frame on the load site. The locking engagement may be rotational, snap-fit, or any other feasible way of fixing, temporarily, the opening of the load capsule to the opening of the load site. The sampling site, recycle site, and harvest site, as will be described below, may have a similar engagement mechanism to the track segments.

The material stored in the load capsules may be in liquid form, or powdered/dehydrated form. Conveyance of material into the load sites may occur via gravity or scooping, with the scooping action being manually performed by the user, mechanically facilitated, or mechanically automated. Load capsules may have a scannable code enabling a user to identify the stored material using a scanning device, such as an external controller. The system may also comprise a scanning device disposed adjacent to the load site in order to confirm the identity of the load capsule prior to conveyance.

The track may comprise a series of segments, with each segment sealable via doors, latches, or gates thereby effecting the quarantining of a given segment against other segments. Each segment may feature its own dedicated set of sensors, growth lights, and load sites.

Segments may be designed for modularity, such that additional segments may be added or removed from the track. Modularity components may include various adapters for adapting a segment of a first diameter to a segment of a second diameter. Segments may be joined and sealed together via dedicated joint fixtures and sealing techniques.

Segments may include cultivation tanks, such as photobioreactors. The segments may be made of glass, fiberglass, acrylic, plastic (such as polyethylene, polypropylene, polycarbonate, and PVC), stainless steel, rock/rocksheet, or concrete. Segment diameter and length may be optimized for light penetration and flow control; segment diameter or other flow area measurements may be changed at key points or throughout the system, either dynamically (via the opening and closing of doors, latches, and gates, the replacing of one pipe segment with another, or telescopically) or passively, by designing the system so that key segments in the system have designated measurements for desired flow properties. Segments may be straight, coiled, or serptentine, and vertical, horizontal, or sloped.

The segments may include safety features, such as pressure relief valves, compressors, and pumps for preventing or alleviating high-pressure zones.

The system may comprise a sampling site, which is a system component capable of capturing algae and algae media at one or more positions along the track segments in order to allow closer inspection of algae properties. The mechanism may include a pipetting mechanism configured to be inserted into track segment walls and a pump to convey the sample into the sampling site, or a valve configured to briefly opened for a set duration and then quickly closed, with the valve allowing flow of the sample into the sampling site. The sampling site may enable human operators to manually test the sample, or may provide more finely-tuned sensors to detect sensor data for computer analysis.

The system may comprise a recycle site, which is a system component, typically a vessel and/or a track segment, which is configured to receive, via a flush cycle, described below, or otherwise, algae growth media, such as algae content, unused nutrients, water, etc. The system may comprise a switch for directing the algae growth media from a track segment toward the recycle site. The recycle site may heat, radiate via UV light, chemically treat, or otherwise manage the algae growth media in order to transform the algae growth media into a form appropriate for reintroduction back into the primary track. In one variation, the recycle site is configured to package the recycled algae growth media into load capsules, which are then reintroduced to the primary track.

In addition to recycling the biological material and cultivation medium, the recycling site, or recycling nodes distributed throughout the system, can capture biogas by collection anaerobic digestion of waste. This biogas can be used as fuel for the system. Additionally, heat generated through physical system processes may be captured for targeting heating of the system. Condensation produced by heating water during sterilization or other system processes may be collected and recycled. Further, the system itself may operate to recycle sewage and other wastewater sources as nutrients for the algae. The algae in turn may treat the wastewater, yielding clean(er) water which may then function as its own product of the system.

The system may comprise a harvesting site, which is a system component, typically a vessel and/or a track segment, which is configured to receive, subsequent to a harvesting cycle, algae content for harvesting. The system may further comprise a switch for directing algae growth media from a track segment toward the harvesting site. Harvesting may be designated as “live” harvesting or “dead” harvesting, as will be described.

The harvesting site may convey the harvested algae content into harvest capsules for removal from the system and subsequent distribution. Harvest capsules may include algae growth media, if a live harvest is desired, or else omit the algae growth media if the algae content is desired dead. Accordingly, the harvesting site may be configured to separate the algae content from the algae growth media via dehydration, filtering, and/or straining. Dehydration may occur via an application of heat by the system and/or an exposure to very high-intensity growth lights. Filtering and/or straining may be accomplished via sieves, centrifugation, flocculation, and/or other mechanisms for exploiting variations in density between the algae content and the algae growth media. Flocculants, such as long-chain polymers generally, or environmentally friendly materials such as chitosan may be utilized to facilitate harvesting by binding the particles into aggregates. The harvesting site may include flocculation and/or centrifugation chambers, washing and drying chambers, etc.

In one version, the system comprises a plurality of harvesting sites and a plurality of harvesting site types. One harvesting site type is a live harvesting site, configured to harvest and preserve the life of algae content. Another harvesting site type is a dead harvesting site, configured to harvest by terminating the life of algae content. Harvesting site types may also be distinguished between whether the algae content is desired for use as fuel, food, or beverage.

The system further comprises a set of growth lights. The growth lights may be fixed to a given range of the light spectrum emittance, or may be configured to undergo changes in light spectrum emittance. The growth lights may be controlled by a user through the external controller via the processor.

The controller may feature a start frequency and stop frequency of light spectrum emittance, with the start frequency signifying the lower frequency of the light spectrum emitted by the lights and the stop frequency signifying the higher frequency of the light spectrum emitted by the lights. The stop frequency may be fixed to the start frequency, such that when the start frequency changes, so too does the stop frequency; or vice versa; or the start and stop frequency may be independently modifiable, such that the range of light spectrum emittance may be contracted or expanded by the controller. Light spectrum parameters of the lights may also be set to vary based on time, such that a first start and stop frequency is set for one period of the day and a second start and stop frequency is set for another period of the day. The start and stop frequency may vary automatically based on a set of equations which calculate the frequencies based on feedback from other sensors, the time during a growth cycle, or the time of day.

Additional or alternative light sources may include natural sunlight, starlight, and moonlight.

Temperature control may be provided via conventional temperature systems and mechanisms, such as heaters or coolers. Temperature control may be administered by direct heating of the physical track, including the piping, the infusion into the system of heated (or cooled) fluid flow, such as water or air.

The system may further comprise a turbidity sensor configured to measure the turbidity (i.e., the light scattered by the algae growth media within the liquid portion) of the algae growth medium within given track segments.

The system may further comprise a set of light sensors configured to detect the intensity and type of light emitted within given track segments.

The system may further comprise a set of temperature sensors configured to measure the temperature within given track segments.

The system may further comprise a set of pH sensors configured to measure the pH within given track segments.

The system may further comprise a set of salinity sensors configured to measure the salinity within given track segments.

The system may further comprise a set of oxygen sensors configured to measure the Oxygen level within given track segments.

### Sensor Data Processing

Sensor data processing may determine whether the algae is dead or alive before instituting a program or protocol. Vibrant colors, such as green, red, or brown, indicate live algae, whereas if limited algae color is detected, the algae is likely dead. Algae spacing and density may also indicate its status, as dead algae tends to clump together and settle down the density gradient. Microscopic examination may reveal whether the cellular structure is intact or shows signs of chloroplast movement (alive) or shriveled or broken (dead). Similarly, fluorometers may detect whether the chlorophyll fluoresce in the presence of light (alive). If the pH is too low, it suggests an over-abundance of carbon dioxide and therefore a lack of consumption of the same (dead). Similarly, a drop in oxygen levels may indicate a lack of photosynthesis (dead). Electrical conductivity patterns of the medium differ in presence of live or dead algae. The rate of nutrient consumption indicates the status as well, with a decrease in consumption indicating that the algae have died.

The controller may be coupled to a user interface, permitting a user to view, research, and mandate one or more parameters, algae status, and system scripts or programs, including one or more cycle types and one or more cultivation types. The user interface may display a set of parameters accompanied by a set of fields, with the user enabled to enter or change the field entry associated with each parameter. The user interface may also enable a user to view all of the load capsules within the load capsule matrix and select one or more for conveyance. The user interface may allow a user to save load capsule groupings in order to make “cocktails” of multiple algae content types, algae medium types, etc.

The system may feature cultivation types, with the cultivation types signifying preferred types of algae cultivation— for example, a first type of cultivation may signify that the algae is intended to be used as fuel, while a second signifies that the algae is intended to be consumed as food, while a third may signify that the algae is intended to be consumed as beverages. Each cultivation type may be associated with a set of default parameters configured to maximize the quality and quantity of the chosen cultivation type. These cultivation types may be selected by a user of the system; after selection, the user interface may display the set of default parameters associated with each cultivation type, such as the intended quantity or mass output of given types of algae, etc., with these associated parameters being customized by the user in order to tweak the desired output.

Cultivation of algae for biomass as fuel may focus in increasing the lipid or carbohydrate content of the algae. Botryococcus braunii, Chlorella, and Nannochloropsis are preferred for their high oil content. The same may be intentionally stressed by the system to further increase their oil content; this stress may be achieved through the reduction of nutrients. Cultivation of algae for food may focus on increasing the nutritional components, such as protein, vitamins, and the elimination of contaminants. Reduction of contaminants are unimportant for algae-as-fuel, but crucial for algae-as-food. Spirulina and Chlorella are preferred for their nutritional value and are considered safe for consumption, and their nutritional output may be increased by reflectively increasing their nutritional intake.

The user interface may feature cycle types, with the cycle types signifying the type of treatment the system will impose on the algae growth media. The cycle types enable the user to control, via the system, the speed of algae cultivation, or else management of the system, such as (live or dead) algae content harvesting, algae content killing, algae growth media flushing, and track sanitization. Some cycle types may include a set of cycles in a sequence. For example, dead algae content harvesting may include the cycles of algae content killing, algae growth media flushing, and finally track sanitization.

The system may feature a harvesting instruction. The harvesting instruction may be split between a “live” harvesting instruction and a “dead” harvesting instruction, with the former designed to keep the algae content alive whereas the latter is designed to terminate algae content life. Harvesting live algae may be relevant for the production of algae-as-food, but for the production of algae-as-fuel, live harvesting is not required. Additionally, the condition of the algae may be determined prior to harvesting— if the algae in an algae-for-food process is determined as alive, live harvesting instructions may be transmitted; otherwise, dead harvesting instructions may be transmitted. Algae for live harvesting may undergo gentle harvesting techniques, such as washing and slow drying in order to preserve cell and nutrient integrity.

The system may feature a kill instruction. The kill instruction is designed to terminate algae cultivation by radically changing one of the system parameters. In one variation, the system may increase the temperature to a level in which algae cannot survive. In another variation, the system may deny introduction of load items or capsules containing a necessary ingredient for algae growth. In yet another variation, the system may increase the growth light intensity beyond algae content life conditions.

In another variation, the system may flush the track by opening waste removal outlets in the track. These waste removal outlets may consist of tangent tracks, with these tangent tracks typically being closed during algae growth cycles. The waste removal tangent tracks may convey flushed algae growth media to the recycle site.

The system may feature a sanitize instruction. Upon selection, the sanitize instruction is designed to clean the track by chemical means, such as with disinfectants like sodium hypochlorite, hydrogen peroxide, quaternary ammonium compounds, or dedicated algaecide cocktails, temperature means, such as hot water, or steam, or with the use of ultraviolet light. In addition, the track segments may be drained for superior surface cleaning. Sanitization instructions may also disengage track segments so that they can be dismantled and cleaned manually and then left to dry to remove any remaining microorganisms. The track segments can also be inspected for rust or other forms of damage before reassembly.

The system is configured to detect, based on sensor feedback, the status of algae content. Particular algae content status facts meriting attention include viability, toxicity, and accordance with cultivation type. Accordance with cultivation type may be detected as lacking if, for example, an incorrect type of algae is being cultivated, as determined by light sensor feedback. As different pH levels are conducive toward the growth of particular algae types, feedback from the pH sensor may indicate whether an undesirable algae content is likely being cultivated. The system may determine whether or not to convey the algae growth media to the recycle site, or the harvesting site based on the aforementioned detection. Further, the system may select whether to transmit the algae growth media to a live or dead harvesting site. Further, the system may select whether the transmit the algae growth media to a food, fuel, or beverage harvesting site.

Each of the recycle site, harvest site, and sampling site may occupy a given portion of track segment or a chamber connected to a track segment with fluid flow engagement.

Typically, the kill, flush, and sanitization instructions are packaged together, such that a single command results in the aforementioned instructions being actuated.

In one variation, the various user interface displays and options may be directed to the system as a whole. In another variation, displays and options may be directed to a given segment. Thus, it may be possible for the user to communicate to the system processor a set of instructions via the external controller, with a first set of instructions for a first segment and a second set of instructions for a second segment.

The system may automatically execute instructions based on detection of various parameters or sensor feedback.

Once a program, script, or cycle is set, the processor may determine whether the conditions of the system, as detected by sensor data, are diverging from preset thresholds native to the program, script, or cycle. Accordingly, the processor may automatically and electronically arrange for the inclusion of additional algae content, nutrients, oxygenation, or any other resource via the load sites or via specialized entryways for the needed resource. The processor may be in instructional communication with the gates and valves to enable the inclusion. The processor may be in instructional communication with the growth lights, heater, and UV light sources, and may control the magnitude, duration, and type, when appropriate, of those resources. Additionally, the processor may be programmed to engage the sampling site for sample capture in order to assist in ensuring that system conditions are as expected or desired.

### BRIEF DESCRIPTION OF THE DRAWINGS

FIG. 1 shows an exemplary system architecture.

FIG. 2a shows an exemplary system process during for actuating kill, flush, and sanitization instructions.

FIG. 2b shows an exemplary system process during for actuating kill, flush, and sanitization instructions.

FIG. 3a shows an exemplary system process during for actuating algae production instructions.

FIG. 3b shows an exemplary system process during for actuating algae production instructions.

FIG. 4 shows an exemplary system process during for harvesting instructions.

FIG. 5 shows an exemplary controller and processor architecture.

### DETAILED DESCRIPTION

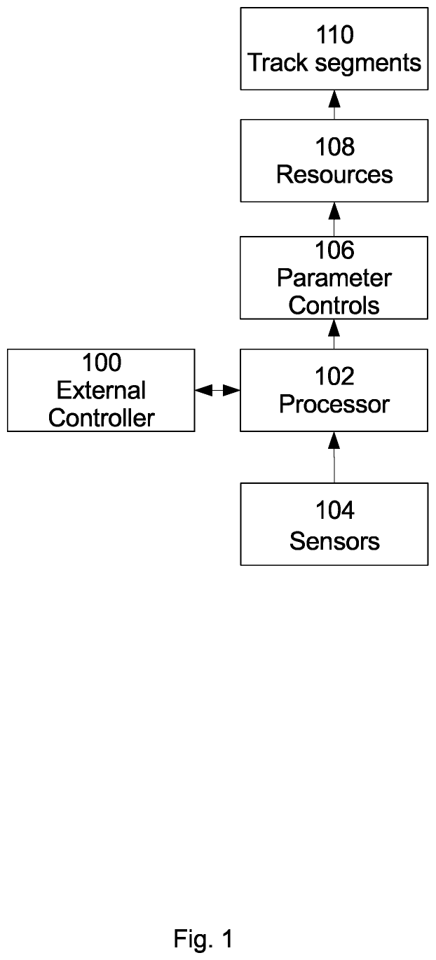

FIG. 1 shows an exemplary system architecture comprising an external controller 100 engaged to a processor 102, with the processor configured to receive data from various sensors 104 and to transmit instructions to various parameter controls 106 based on instructions from the external controller as well instructions derived from scripts with sensor data as the input. Parameter controls include heaters, growth lights, UV light sources, and gates, doors, valves, and nozzles enabling the flow of materials and resources 108 onto the track segments 110.

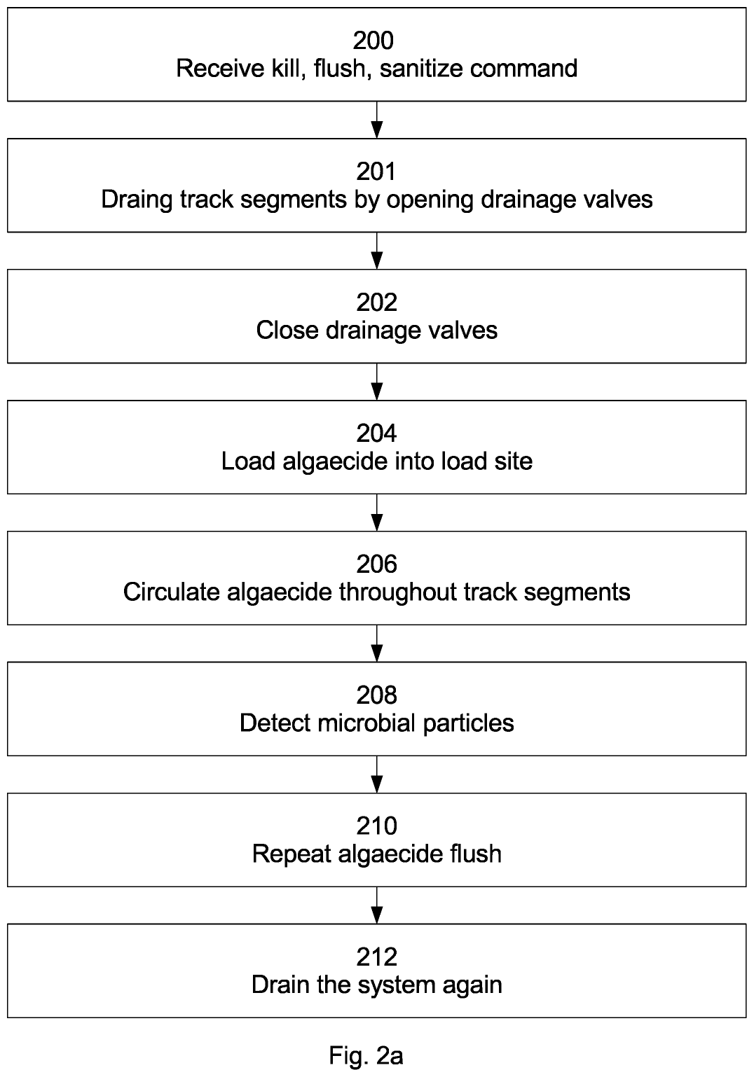

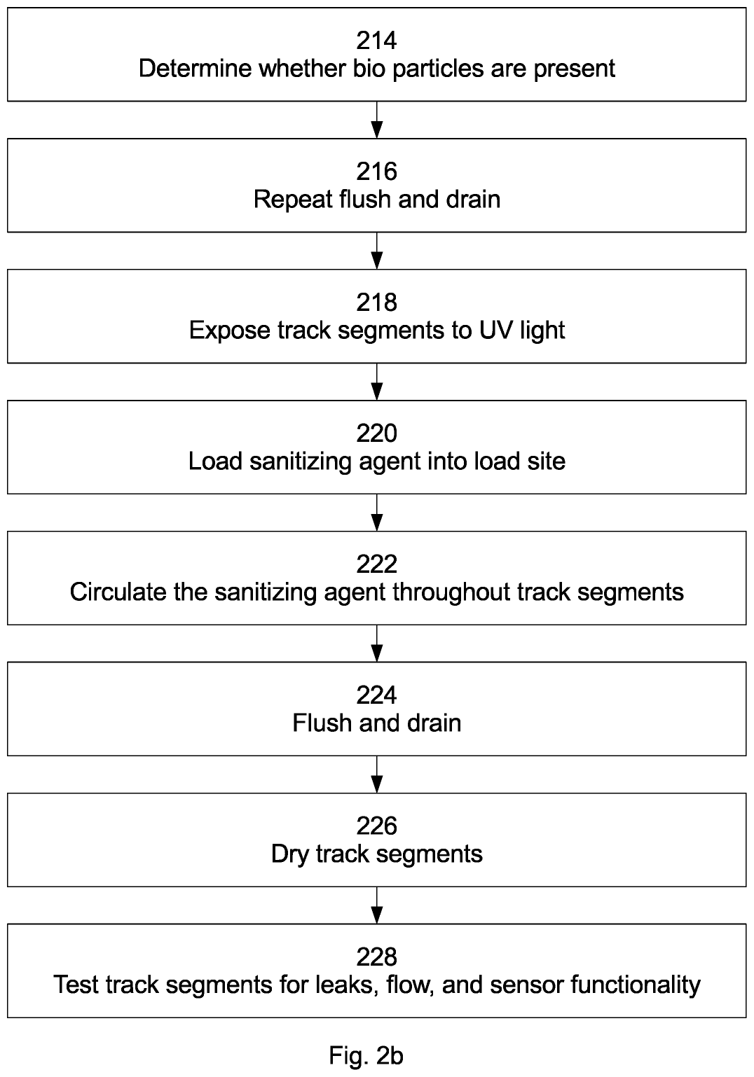

As shown in FIGS. 2a-2b, in an exemplary kill, flush, and sanitize instructions package, a single command 200 actuates the following steps: drain the track segments by opening drainage valves, doors, or gates 201, close the drainage valves 202, load algaecide into the load site 204, circulate the algaecide throughout the track segments 206, detect using bio sensors whether microbial particles are live 208, and if so, repeat algaecide flush 210. Drain the system again 212, determine whether bio particles are still present 214, and if so, repeat flush and drain step 216. Expose track segments to UV light 218, load sanitizing agent into the load site 220, and circulate the sanitizing agent throughout the track segments 222, flush and drain 224, and passively or actively dry the track segments 226. Finally, test the track segments for leaks, flow, and sensor functionality with a water wash 228.

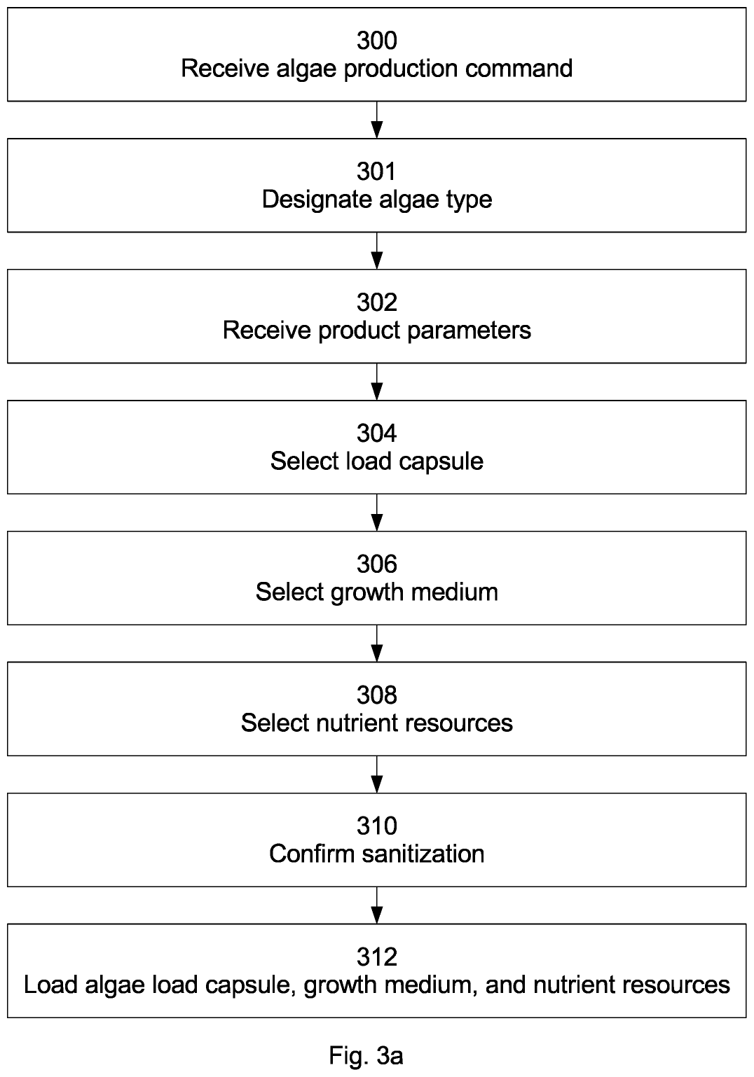

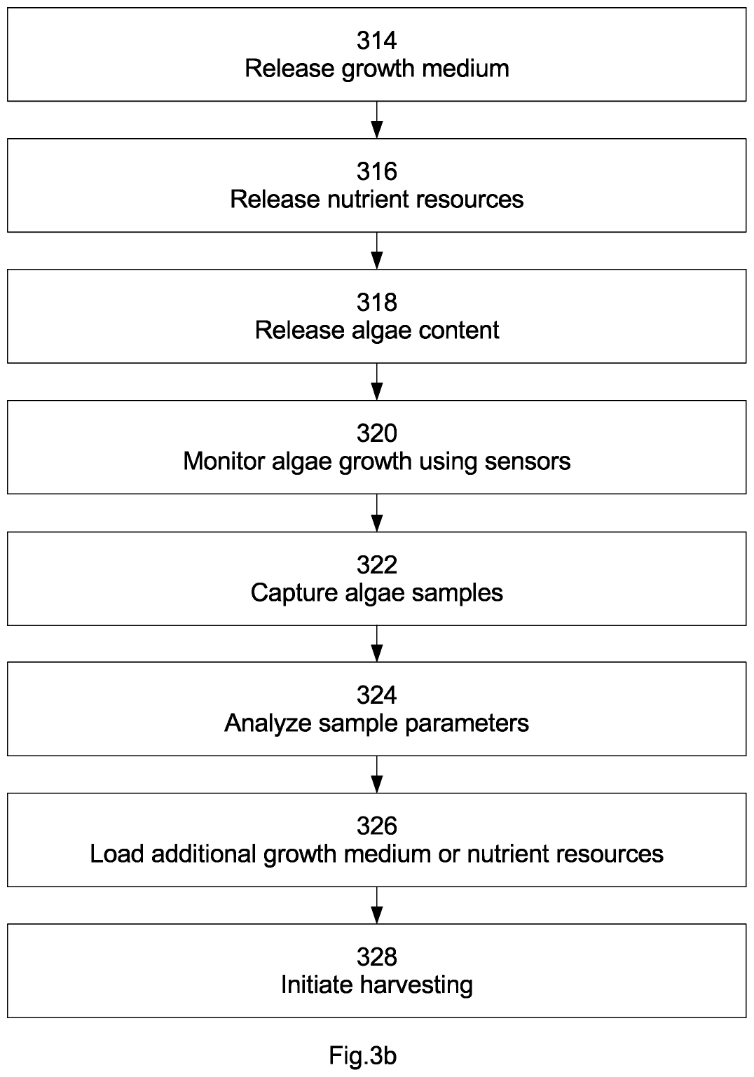

As shown in FIGS. 3a-3b, in an exemplary algae production instructions package, a single command 300 actuates the following steps: identify whether the desired product is algae-for-fuel or algae-for-food 301, receive product parameters further designating product qualities 302, select a load capsule containing the algae most capable of yielding the product type and parameters based on an algae production database 304, select a growth medium most conducive to growing the selected algae 306, select nutrient resources most conducive to augmenting the growth potential and obtaining the product parameters 308, confirm that the track segments has been sanitized 310, load the algae load capsule, growth medium, and nutrient resources at the load site 312, release the growth medium 314, release the nutrient resources, either all at once or gradually, into the growth medium 316, release the algae into the growth medium 318; monitor algae growth using sensors 320, capture algae samples 322, analyze sample parameters 324, load additional growth medium or nutrient resources depending on the analyzed sample parameters 326, initiate harvesting when lipid or protein/carbohydrate levels at peak 328.

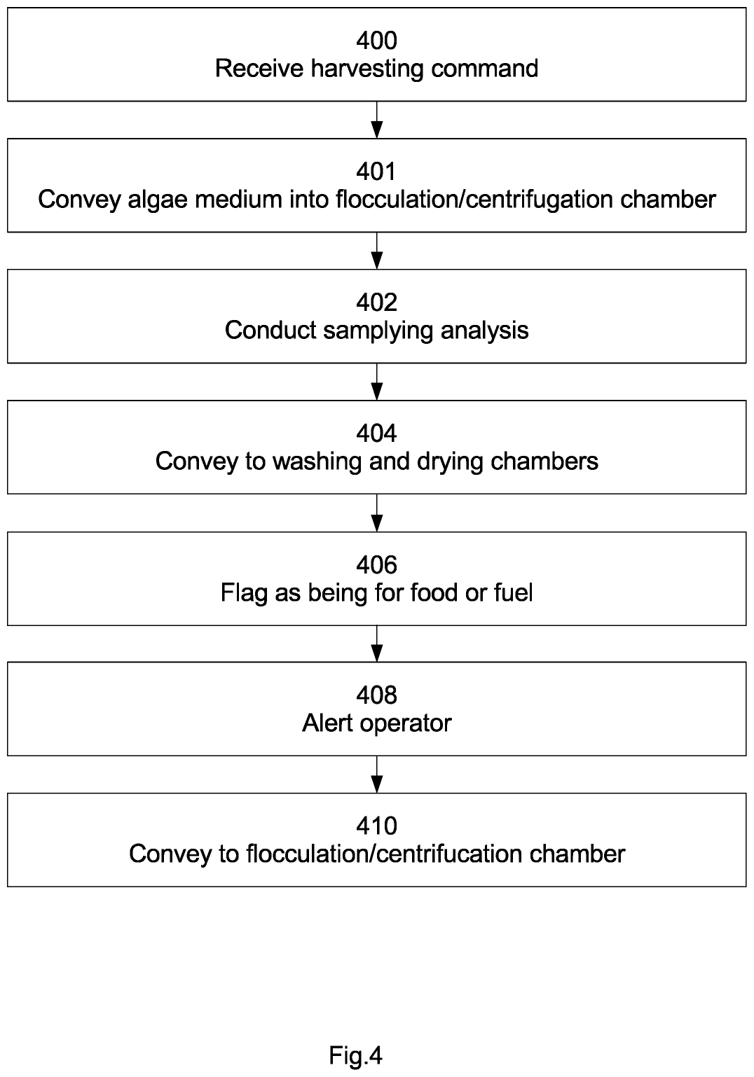

As shown in FIG. 4, in an exemplary harvesting instructions package, a single command 400 may actuate the following steps: if algae is intended for fuel, convey the algae media into a flocculation or centrifugation chamber for fuel harvesting 401, or if algae is intended for food, conduct sampling analysis 402. If the algae is live and not contaminated, convey the same to the washing and drying chambers for live harvesting 404. If the algae is dead but not contaminated, flag the algae as being optionally for food or fuel 406 and alert an operator 408. If the algae is contaminated, convey the same to the flocculation or centrifugation chambers for fuel harvesting 410.

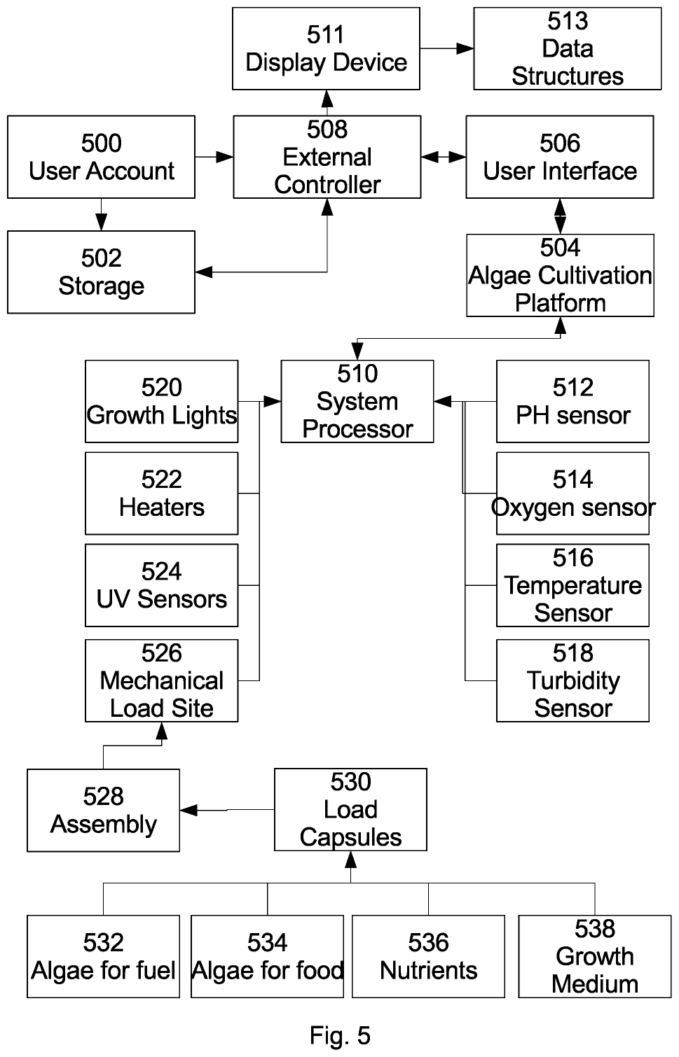

FIG. 5 shows an exemplary controller and processor architecture comprising a user account 500 coupled to a storage medium including backup online storage 502. The user account is configured to operate to allow access to an algae cultivation platform 504, which provides a user interface 506 via an external controller 508 for accessing a system processor 510 (such as, for example, a Raspberry Pi). The system processor may also be embedded in the external controller, or wirelessly connected to the external controller via Bluetooth or SSH (secure shell) network protocol. The external controller may provide for the viewing of system parameters, processors, and an interface via a display device 511, with the system parameters shown in tabulation, graphs, plots, or other viewable data structures 513. The system processor may be in informational contact with a variety of sensors, including a pH sensor 512, an oxygen sensor 514, a temperature sensor 516, and a turbidity sensor 518. The system processor may also be in instructional contact with growth lights 520, heaters 522, UV sensors 524, and a mechanical load site 526. The load site in turn is engaged mechanically and electronically to a load capsule and reservoir assembly 528, with the load capsule and reservoir assembly comprising a matrix of load capsules 530 containing various algae, including algae for fuel 532 and algae for food 534, and various nutrients 536 and growth mediums 538.

1. A system comprising a set of track segments, a set of load sites, a set of load capsules, a set of liquid reservoirs, growth lights, a set of sensors, an external controller, a processor, a heater, a sampling site, a recycle site, a harvest site, and a UV light source;
a. with the set of load capsules configured to engage with the set of load sites;
b. with the load sites configured to convey content from the load capsules onto the track segments;
c. with the set of load capsules comprising a first load capsule containing algae of a first type, a second load capsule containing algae of a second type, a third load capsule containing growth medium of a first type, a fourth load capsule containing growth medium of a second type, a fifth load capsule containing nutrients of a first type, and a sixth load capsule containing nutrients of a second type;
i. with the algae of the first type being algae for fuel and the algae of the second type being algae for food;
ii. with the growth medium of the first type having a composition conducive to growing algae of the first type and the growth medium of the second type having a composition conducive to growing algae of the second type;
iii. with the nutrients of the first type being used to feed algae of the first type and the nutrients of the second type being used to feed algae of the second type;
d. with the set of liquid reservoirs comprising a first liquid reservoir containing liquid having a lower pH than liquid in a second liquid reservoir;
e. with the track segments made of glass, plastic, stainless steel, or concrete and configured to provide and control circulation of liquid, algae, growth medium, and nutrients via pumps and valves;
f. with the track segments having transparent or semi-transparent walls to enable light penetration;
g. with the growth lights being capable of emitting and varying the emittance of a plurality of light frequencies or comprising a plurality of growth light types; with a first growth light type having a higher light frequency than a second growth light type;
h. with the external controller comprising one or more computers, a display device, and one or more input devices;
i. with the external controller being configured to receive instructions from a user pertaining to system parameters and programs and view sensor data;
i. with the programs including an algae selection program or a final product selection program and a recycle or harvest cycle;
ii. with the system parameters including temperature, pH levels, and algae density;
j. with the processor configured to receive sensor data and relay the sensor data to the external controller;
k. with the processor configured to determine whether the sensor data diverges from preset thresholds, and if so, seal or unseal a subset of track segments, activate the load site to dispense lacking nutrients, distribute balancing pH liquid flow, adjust the heater, or adjust the growth lights;
l. with the processor configured to seal or unseal a subset of track segments, activate the load site to dispense lacking nutrients, distribute balancing pH liquid flow, adjust the heater, or adjust the growth lights based on a programmatic selection by the user via the external controller;
m. with the sampling site configured to engage with the track segments and capture a sample of algae and medium via pipetting or briefly opening a valve;
n. with the recycle site configured to radiate the algae content via the UV light source, cook the algae content via the heater, and sanitize the algae content via chemical sanitizers, and then to return the algae content to the track segments to be used as nutrients for a subsequent cultivation cycle;
o. with the harvest site configured to separate the algae content from the growth medium via dehydration or filtering, and then to package the harvested algae content.

2. A system comprising a set of track segments, a set of load sites, a set of load capsules, growth lights, a set of sensors, an external controller, a processor, a heater, a harvest site, and a UV light source;
a. with the set of load capsules configured to engage with the set of load sites;
b. with the load sites configured to convey content from the load capsules onto the track segments;
c. with the track segments configured to provide and control circulation of liquid, algae, growth medium, and nutrients;
d. with the external controller comprising one or more computers, a display device, and one or more input devices;
e. with the external controller being configured to receive instructions from a user pertaining to system parameters and programs and view sensor data;
i. with the system parameters including temperature, pH levels, or algae density;
f. with the processor configured to seal or unseal a subset of track segments, activate the load site to dispense lacking nutrients, distribute balancing pH liquid flow, adjust the heater, or adjust the growth lights;
g. with the harvest site configured to separate the algae content from the growth medium via dehydration or filtering.

3. The system of claim 2, with the set of load capsules comprising a load capsule containing algae of a first type, and a load capsule containing algae of a second type.

4. The system of claim 3, with the algae of the first type being algae for fuel and the algae of the second type being algae for food.

5. The system of claim 2, with the set of load capsules comprising a load capsule containing growth medium of a first type and a load capsule containing growth medium of a second type.

6. The system of claim 5, with the growth medium of the first type having a composition conducive to growing algae for food and the growth medium of the second type having a composition conducive to growing algae for fuel.

7. The system of claim 2, with the set of load capsules comprising a load capsule containing nutrients of a first type and a load capsule containing nutrients of a second type.

8. The system of claim 2, additionally comprising a set of liquid reservoirs, with the set of liquid reservoirs comprising a first liquid reservoir containing liquid having a lower pH than liquid in a second liquid reservoir.

9. The system of claim 2, with the growth lights being capable of emitting and varying the emittance of a plurality of light frequencies or comprising a plurality of growth light types, with a first growth light type having a higher light frequency than a second growth light type.

10. The system of claim 2, with the programs including an algae selection program or a final product selection program.

11. The system of claim 2, with the programs including a recycle or harvest cycle program.

12. The system of claim 2, additionally comprising a sampling site, with the sampling site configured to engage with the track segments and capture a sample of algae and medium via pipetting or briefly opening a valve.

13. The system of claim 2, additionally comprising a recycle site, with the recycle site configured to radiate the algae content via the UV light source, cook the algae content via the heater, and sanitize the algae content via the chemical sanitizers, and then to return the algae content to the track segments to be used as nutrients for a subsequent cultivation cycle.

14. A system comprising a set of track segments, a set of load sites, growth lights, a set of sensors, a processor, a heater, a harvest site;
a. with the load sites configured to convey content onto the track segments;
b. with the track segments configured to provide and control circulation of liquid, algae, growth medium, and nutrients;
c. with the processor configured to, upon detecting sensor data divergences or upon reaching programmatic thresholds, seal or unseal a subset of track segments, activate the load site to dispense nutrients, distribute balancing pH liquid flow, adjust the heater, or adjust the growth lights;
d. with the harvest site configured to separate the algae content from the growth medium via dehydration or filtering.

15. The system of claim 14, with the load sites configured to convey content via engaging with or receiving load capsules,

16. The system of claim 15, with the load capsules containing algae content, algae growth medium, or nutrients,
a. The system of claim 15, additionally comprising an external controller, with the external controller comprising one or more computers, a display device, and one or more input devices;
b. with the external controller being configured to receive instructions from a user pertaining to system parameters and programs and view sensor data.

17. The system of claim 16, with the system parameters including temperature, pH levels, or algae density.

18. The system of claim 15, with the programmatic thresholds corresponding to when algae content is at peak lipid or carbohydrate production.

19. The system of claim 15, with sensor data divergences being pH levels or algae density which indicating that algae content is dead.

20. The system of claim 15, additionally comprising a recycle site and a UV light source, with the recycle site configured to radiate the algae content via the UV light source, cook the algae content via the heater, and sanitize the algae content via chemical sanitizers, and then to return the algae content to the track segments to be used as nutrients for a subsequent cultivation cycle.
